(19) United States
(12) Patent Application Publication
(10) Pub. No.: US 2003/0004470 A1
(43) Pub. Date: Jan. 2, 2003

(54) Method and apparatus for managing temperature and flow of medical fluids

(76) Inventors: Barry Hickerson, Modesto, CA (US); Laura McIntosh, Palo Alto, CA (US)

Correspondence Address: Laura McIntosh, 832 Kipling St # 3, Palo Alto, CA, 94301, US

(21) Appl. No.: 10/187,107

(22) Filed: Jul. 1, 2002

Related U.S. Application Data
(60) Provisional application No. 60/302,921, filed on Jul. 2, 2001.

(51) Int. Cl. A61M 5/00

(52) U.S. Cl. 604/251; 604/113; 604/508

(57) ABSTRACT

Methods and apparatus are described for controlling the temperature and flow rate of fluids being delivered to patients. A control unit is disclosed that interfaces with specialized fluid administration set apparatus. The control unit provides power, user interface and integrated pumping and heat exchange apparatus that interfaces with a cartridge on the fluid administration set. The fluid administration set is a disposable apparatus while the control unit is a reusable device. The entire system, due to its portability and engineering features, is adapted for use either in-hospital or in the pre-hospital, emergency setting.

### RELATED APPLICATIONS

The present application claims priority benefit under 35 USC §119(e) from U. S. Provisional Application No. 60/302,921 filed Jul. 2, 2001, entitled “METHOD AND APPARATUS FOR MANAGING TEMPERATURE AND FLOW OF MEDICAL FLUIDS”, which is incorporated herein by reference.

### FIELD OF THE INVENTION

The field of this invention is fluid administration for medical purposes.

### BACKGROUND OF THE INVENTION

Fluid administration is a common occurrence during medical care of animals. Such fluids include blood and blood products, hydration fluids, solutions for lavage and irrigation, and fluid drips for administration of pharmaceuticals. Medical research indicates that administration of fluids at normal body temperature (37° C.) is desirable, and that administration of fluids cooler than body temperature can result in medical complications. Current technology for fluid warming is cumbersome and inconvenient, or very expensive, so medical personnel usually do not make the effort, take the time, or incur the expense to warm fluids before use unless the procedure is critical to patient care (such as during major surgery).

There is also a need for accurate and time-sensitive administration of specific fluid volumes. Sometimes large quantities of liquids are necessary, such as treating dehydration or administering blood during severe trauma, and must be given quickly. At other times the quantities are smaller and more precise, such as when pharmaceuticals are administered. Medical personnel who administer large volumes of fluids, such as nurses and paramedics, do not have the ability to give patients partial bags of fluids accurately, nor can they afford to take the time to constantly monitor patients to ensure proper flow rate. Most often these caregivers estimate both quantity and flow rate of fluid administration because they are unable to specify optimal amounts and rates. Fluid management is used not only to administer fluids to patients. Fluids are also used as carriers of pressure and temperature for medical purposes. A hot water bottle is a familiar example of a fluid within a reservoir used to carry heat to a patient.

The current art in fluid administration has not fully addressed the need for temperature management of those fluids. Because fluids are often given to a patient in an emergency setting, no time is taken to warm the fluids, which are often stored at cold or freezing temperatures, before they are delivered to the patient. When warming is attempted, it may be in the form of a caregiver sitting on a bag of fluid to use their body temperature to warm the fluid. The prior patent art describes many systems for fluid administration. Many of these patents describe systems with pumps and many of these systems describe systems with different types of heaters or cooling devices, but none of these systems provide both controlled pumping and temperature management of pre-filled reservoirs of sterile fluids prior to delivery in a portable design relevant to both field and hospital use.

From the foregoing, it can be appreciated that there is a need for a fluid administration system that is both portable, quick to deploy, and easy too use while delivering fluids to patient at controlled flow rates and controlled temperatures. The systems of the prior art do not fulfill these requirements.

### SUMMARY OF THE INVENTION

The invention is an apparatus and method for managing the delivery of fluids for medical purposes at physiologically appropriate temperatures. The apparatus described herein is a fluid administration system that will conveniently, quickly, and accurately manage the temperature and flow rate of medical fluids at the same time. The fluids are delivered to animals, or more specifically mammals, or more specifically yet, humans. The animal to which the fluids are delivered is a patient.

The system comprises a control unit that contains the mechanism for heat exchange and pumping of a fluid that is contained within a disposable fluid administration set. The control unit is designed to operate on battery power to facilitate its use for emergency personnel, but can be plugged into standard wall outlets or 12-volt vehicle outlets for recharging or regular use. The ability of emergency personnel to administer temperature and rate-controlled fluids will greatly enhance field treatment of medical patients. The fluid administration set, further comprised by the system, is a disposable element that attaches at one end to a pre-existing fluid source and has a standard medical attachment at the other end for connecting to the patient. Within the length of the fluid administration set are a heat exchange and pumping cartridge, and the standard fluid line components such as a drip chamber and drug injection ports.

The apparatus includes a pumping mechanism for fluid propulsion, a temperature control device and heat transfer element that heats or cools the fluid, one or more receptacles that accept and hold in place a cartridge containing fluid. The cartridge optionally contains an information key enabled, for example by shape or electronics, that controls the temperature at which the apparatus will heat or cool the fluid, operator controls, and an energy source. The information key, on the cartridge, communicates information to the controller through a key interface on the controller. The pumping mechanism can be set to move specific volumes of fluid at a specified rate. The pump can operate at a low enough pressure and shear rates so as to deliver blood products without cell lysis. The temperature control unit heats or cools fluid to the specified temperature as the fluid moves through a separable cartridge within the receptacle. The desired fluid temperature will be achievable at all rates of flow as provided by the pumping mechanism. The energy source preferably incorporates a rechargeable battery system for portability or safety, although the unit can also be plugged into a standard wall socket. Use of chemical reactions as a heat source is also adaptable as a heating or cooling source. Operator controls determine flow rate, and heater on/off state. Other features include indicator lights for failure to achieve indicated flow, low battery power, pump shutoff if specified temperature is not achieved in cartridge, and overall proper functioning equipment status. The control unit also contains a data access port for monitoring parameters by outside equipment, or for specifying a sequence of temperature/pumping parameters by external equipment like a computer or data recorder.

The method of the invention includes infusing a patient with fluids at a controlled rate that is determined by an active pumping system while controlling the temperature of the fluid through the use of an active heating or cooling system. The method of the invention further includes the use of a cartridge that is integral to the disposable fluid administration set and that interfaces with a controller, the cartridge performing heat transfer, pumping and parameter setting functions. The disposable fluid administration set comprises a line of tubing connected to a cartridge containing a pumping mechanism and heat exchanger. The cartridge fits into the separate control unit. The cartridge has a heat exchange surface that interfaces with a heater/cooler contained within the control unit, a pumping mechanism that interfaces with the pump contained within the control unit, and a channel through which the fluid will flow. The tubing is affixed onto the cartridge, which further contains a channel of sufficient length to provide the residence time needed for warming or cooling to occur at the rate of volume flow provided by the control unit pump or gravity. The cartridge selects the fluid temperature from the control unit by means of either mechanical or electrical signaling contained within the cartridge and detected by the control unit. The cartridge or the tubing optionally contains sensing elements detectable by the control unit for temperature and flow parameters, and the tubing may contain unidirectional valves. The end of the tubing from which flow is drawn shall have a connection appropriate for containers of fluids intended for intravenous administration to any mammal, and the end of the tubing toward which fluids are pumped shall have a connection appropriate to the site of administration of intravenous fluids to any mammal. The fluid administration set components should, preferably, not be separated from each other. The fluid to be delivered may be composed of any solution, including hydration fluids, blood products, crystalline or colloidal solutions, and pharmaceuticals.

The control unit of this system may also be used to deliver warmed or cooled fluids to a therapy pad for medical uses such as muscle and joint therapy, dermatological interventions, and pain relief. The availability of a fluid therapy system with temperature controls that can be safely manipulated by both medical caregivers and the public for therapeutic purposes will greatly increase treatment options for a number of medical conditions.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 shows a perspective view of a control unit of an embodiment of the present invention. The control unit provides power, fluid pumping, fluid heating, user interface, and control functions for the fluid administration set through interface with the cartridge, according to aspects of an embodiment of the invention;

FIG. 2 shows a perspective view of the disposable fluid administration set of an embodiment of the present invention. The disposable fluid administration set interfaces with the control unit and handles the fluid administered to the patient, according to aspects of an embodiment of the invention.

### DETAILED DESCRIPTION OF THE INVENTION

In accordance with one embodiment of the present invention, one fluid administration system is described herein. In order to fully specify this preferred design, various embodiment specific details are set forth, such as the number and makeup of the components in the fluid administration set and control unit. It should be understood, however that these details are provided only to illustrate one embodiment, and are not intended to limit the scope of the present invention.

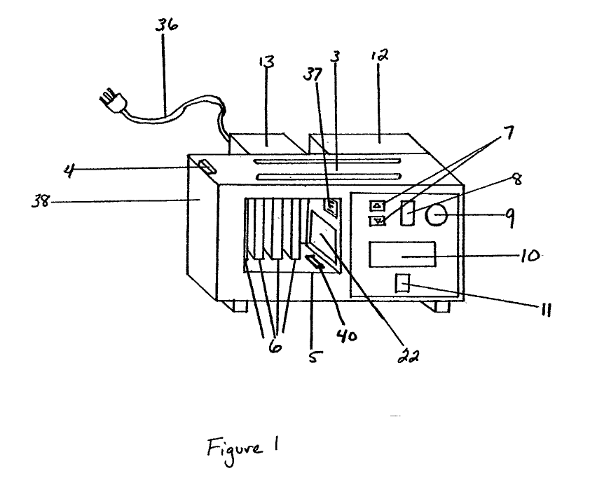

FIG. 1 illustrates a control unit 1, of an embodiment of the invention, comprising a case 38, a handle 3, an attachment clip 4, a receptacle 5, a plurality of temperature elements 6, a fluid pump 22, a plurality of flow rate controls 7, a pump power switch 8, a plurality of indicator lights 9, a display 10, a heater power switch 11, a battery 12, a power supply 13, and a power cord 36, a key interface 37, a chassis 39 (not shown), and a plurality of cartridge locking mechanisms 40.

In this embodiment, the case 38 surrounds, protects, and is mechanically affixed to the chassis 39. The chassis 39 and the case 38 serve as the physical mounts to which all components are affixed. The handle 3 is affixed to the top surface of the case 38 and may be optionally recessed, extendable, or both. The receptacle 5 is affixed to the front panel, top or one of the sides of the case 38 and serves as an opening through which a cartridge 19 (not shown) is inserted. The heating elements 6 are affixed to the chassis 39 and are exposed within the receptacle 5 and come into intimate reversible contact with mating surfaces on the cartridge 19. The fluid pump 22 is affixed to either the chassis or the case and, preferably, at least some part of the pump 22 contacts at least a portion of the cartridge 19 within the receptacle 5. The flow rate controls 7, the pump power switch 8, the indicator lights 9, the display 10 and the heater power switch 11 are all affixed to the front, top or side panels of the case 38. The attachment clip 4 is affixed to the top, front, side, back or bottom of the case 38. The battery 12 is reversibly affixed to either the case or the chassis 39. The power supply 13 is affixed to the chassis 39 or the case 38. The power cord 36 is mechanically and electrically affixed to the power supply 13. The key interface 37 is affixed to the case 38 or chassis 39 so that it is exposed within the receptacle 5, as are the cartridge locking mechanisms 40.

Referring to FIG. 1, the controller 1 case 38 serves to protect the controller 1 from the environment and provide a mounting platform for the controller 1 components. The chassis 39 is affixed to the interior of the case 38 and provides a solid mechanical and electrical platform on which to mount those components that are not directly affixed to the case 38. The attachment clip 4 provides mechanical and electrical connections for an accessory unit or module (not shown) to provide additional functions not specified here or to attach the control unit 1 to a stand or bracket (not shown).

The receptacle 5 provides a readily identifiable and robust attachment port into which the fluid cartridge 19 (not shown) is inserted. The cartridge locking mechanisms 40 are provided within the receptacle 5 to positively and reversibly engage with locking elements on the fluid cartridge 19. Disposed within the receptacle 5 are temperature elements 6 into which the fluid cartridge 19 engage so as to provide heat exchange between the temperature elements 6 and the fluid 20 within the cartridge 19. The temperature elements 6 are preferably fabricated from metals such as but not limited to aluminum, stainless steel, copper, and the like, or other materials with high heat transfer capabilities. The temperature elements 6 are configured to maximize surface area and thus to maximize heat transfer. Such maximized surface area configurations include multiple fingers, flanges, projections, and the like. Also within the receptacle 5 is a pump 22 (only the surface is shown) that will interface with a diaphragm, tubing, or other pumping surface on the fluid cartridge 19. Pump 22 can, for example, be a roller pump, a piston pump, a cam pump, a rocker arm, or the like. The operator can specify flow rate with increase/decrease buttons 7, and turn the pump on and off with on/off switch 8 if gravity flow is preferred. An indicator light 9 or other display allows a visual check at a distance by an operator to see if the unit is operating properly. There is also the alphanumeric display 10 of both flow and temperature for more precise monitoring of the current settings. The alphanumeric display 10 may be of any type such as but not limited to LED, LCD, TFT, plasma, CRT and the like. The temperature elements 6 can be manually turned off with the on/off switch 11 if desired or they may be operated by a logic device or computer (not shown) within the control unit 1.

The side of the control unit 1 opposite the accessory attachment clip 4 includes accommodations for the power supply 13. The battery pack 12 is disposed so that it is easily accessible even when units are stacked. The battery pack 12 is optionally rechargeable and replaceable. Battery chemistries typical of the state of the art are appropriate for this application. Such battery chemistries include but are not limited to lead acid, nickel metal hydride, lithium ion and the like. A power supply 13 with the power cord 36 is disposed near to the battery 12 and serves as an internal recharging system for the battery 12. The power supply 13 is preferably configured to accept both 110 VAC US and 240 volt-AC international power configurations as well as the 12-volt DC power that is available on most motor vehicles. Both batteries 12 and power supply 13 may be incorporated into the body of the case 38 or be separable.

All switches are preferably backlit and color-coded to help identify which features are on or off, and to help operators distinguish features quickly and easily. The display 10 on the control unit provides for easy confirmation of temperature and flow in both high and low light conditions.

The control unit 1 is part of a self-contained modular system designed to allow a caregiver to deliver temperature-controlled fluids 20 at a specified rate for medical applications. These medical applications include fluids for intravenous administration, as well as fluids used to wash tissues or surfaces (lavage and irrigation). Within this context, having fluids at a specified temperature, and flowing at a specified rate, is desirable for health and comfort of the patient. The specific embodiment presented here is not the only form of the device. In another embodiment, the control unit 1 comprises a reusable case 38 containing the elements necessary to manage fluid supplied through the disposable cartridge 19. The control unit 1 is designed to be robust enough for field use by emergency personnel, and is also appropriate for use within any medical facility. Such robustness requires that the device can withstand mishandling and severe impact forces. Designs for robustness preferably include rubberized coatings on the case 38 and high strength connections between the case 38 the chassis 39 and all components. Additionally, the componentry is preferably of high reliability type and able to withstand high G loads. The control unit 1 is self-contained and portable, weighing approximately less than 20 pounds and capable of operation when not connected to an external power source.

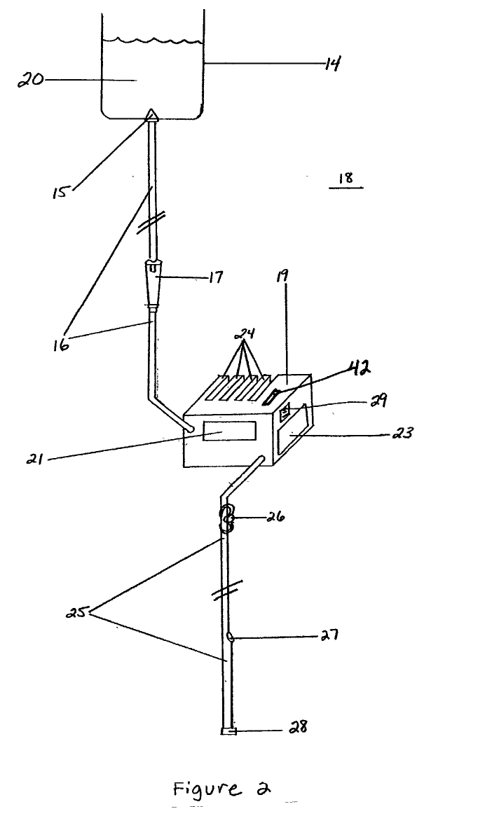

FIG. 2 illustrates a disposable fluid administration set 18 of an embodiment of the invention. The fluid administration set 18 comprises a reservoir 14 containing a fluid 20. The fluid administration set further comprises a spike 15, a length of inflow tubing 16, an optional drip chamber 17, the cartridge 19, a length of outflow tubing 25, an optional roller clamp 26, an injection port 27, and a Luer adapter 28. The cartridge 19 further comprises a label 21, a pumping element 23, a plurality of heat exchange surfaces 24, an information key 29, and a locking structure 42.

As shown in FIG. 2, at the point of contact with a reservoir 14, the fluid administration set 18 comprises the spike 15 to penetrate the fluid reservoir 14. The inflow tubing 16 is affixed to the spike 15 at one end and to the cartridge 19 at the other end. The optional drip chamber 17 is affixed at an intermediate point to the inflow tubing 16 to allow operator verification of fluid 20 flow by visual examination. The cartridge 19, into which the fluid 20 flows, will provide a frame for pumping and heating or cooling the fluid. Each cartridge 19 has a label 21 affixed on the front surface. The label 21 describes the characteristics of the cartridge 19, particularly the temperature at which fluid is to be delivered as set by the cartridge 19. Within the frame, case or outer surface of the cartridge 19 and exposed to the exterior is a pumping element 23, such as a diaphragm, rotary component, or collapsible volumetric component such as tubing. The heat exchange surfaces 24 are affixed to the cartridge 19 outer surface on the top, bottom, sides or back. The information key 29 is affixed to the outer surface of the cartridge 19. The locking structure 42 is a feature either affixed to or integral to the outer surface of said cartridge 19. The cartridge is affixed to the inlet side of the length of outlet tubing 25. The distal end of the outlet tubing 25 is affixed to the Luer port 28. The roller clamp 26 is reversibly attached around, and constricts, the outlet tubing 25 to a variable degree at some intermediate location. The injection port 27 is affixed at an intermediate location on the outlet tubing 25.

Referring to FIGS. 1 and 2, the pumping element 23, interacting with the pump 22, contained within the control unit 1, moves the fluid 20 through the cartridge 19, and fluid administration set 18. The cartridge 19 is inserted into the receptacle 5 of the control unit 1 and interfaces with the heat exchange surfaces 6 within the receptacle 5 of the control unit 1 through the plurality of fins or other high surface area structures 24. These temperature element surfaces 24 become in intimate contact with similar temperature elements 6 within the receptacle 5 of the control unit 1. Ideally, the temperature elements 6 are reverse or mirror image projections of the temperature element surfaces 24 and allow for reversible interlocking or intimate contact between said surfaces.

The fluid 20 passes through the cartridge 19, is set to a specified temperature and flow rate by the control unit 1, then exits the cartridge 19 through outflow tubing 25 to a patient 30 (not shown). There will be contained, within the outflow tubing, various points of intervention for a caregiver, such as an optional roller clamp 26 and the at least one injection port 27 for pharmaceuticals. The outflow tubing 25 terminates in the Luer adaptor 28, other connection commonly used for patient care, or the like.

The present invention is a modular system comprising a reusable, self-contained, controller 1 and a disposable fluid administration set 18 designed to allow a caregiver to deliver temperature-controlled fluids at a specified rate within a medical context. This includes fluids for intravenous administration, as well as fluids used to wash tissues or surfaces (lavage and irrigation). Within this context, having fluids at a specified temperature, and flowing at a specified rate, is desirable for health and comfort of the patient. The specific embodiment presented here is not the only possible form of the device. The current embodiment of the fluid administration set is the fluid inflow line 16, the structurally rigid or semi-rigid cartridge 19 that inserts into the control unit 1 within this modular system, and the outflow line 25 that delivers the fluid 20 to the patient 30. This fluid administration set is designed to be robust enough for field use by emergency personnel, and is also appropriate for use within any medical facility.

The present invention includes methods of administering fluids 20 to a patient 30 that are delivered at a set rate determined by active pumping and at a set temperature determined by control values and enabled through the use of heaters and/or cooling devices that interface with the fluid 20 through the cartridge 19.

The fluid inflow line 16 and the fluid outflow tubing 25 are preferably fabricated as extrusions of polymers such as but not limited to PVC, polyethylene, polypropylene, polyurethane and the like. The extrusions comprise an inlet end and an outlet end. The extrusions further comprise a wall and an inner lumen that is isolated from the environment by the wall. The inside diameters of the tubing typically ranges from less than 1 mm to over 20 mm. The extrusions used in fabricating the tubing 16 and 26 may optionally comprise insulating layers of foam, air, or the like to minimize heat loss to the environment.

The spike 15, drip chamber 17, injection port 27, the roller clamp 26, and the Luer fitting 28 are preferably fabricated by injection molding from polymers such as but not limited to PVC, PET, polyethylene, polypropylene, polycarbonate and the like. The hardness of the polymers used in these parts is generally higher than the hardness of the polymers used to fabricate the tubing 16 and 25. The fluid injection port 27 is configured as a T or Y configuration with the inlet line 16 affixed to one port, the outlet line 25 affixed to another port and the third port used in conjunction with a valve (such as a stopcock) or a seal, which is typically made of an elastomer, to allow fluid to be injected without leakage once the fluid source is removed.

The fluid administration apparatus and method is appropriate for any medical fluid 20, and can be used to deliver fluids for any purpose. The cartridge 19 has a pumping mechanism 23 for fluid circulation, a temperature sensor and/or a flow sensor or information key 29 to provide feedback or control input to the control unit, shaped protrusions 24 with heat exchange surfaces that interface with similar protrusions within the control unit 1. These protrusions 24 maximize heat transfer between temperature elements in the control unit 1 and the cartridge 19. The cartridge 19 also comprises a locking structure 42 that allows locking of the cartridge 19 when it is fully inserted into control unit 1 and which then reversibly engages the locks 40 on the control unit 1 to lock the cartridge 19 into place and enhance contact between the two heat exchange surfaces. Such locking mechanism 40 and 42 could include electromagnets in the control unit 1 that pull on ferrous magnetic strips on the cartridge 19, or other mechanical means such as cams, spring clips, or thee like.

The information key 29 pre-sets the temperature and/or flow rate to which the control unit will maintain the temperature and flow rate of the fluid being delivered. The information key 29 is selected by the user or caregiver based on the label 21 visible on the cartridge 19 or other part of the fluid administration set 18. The information key 29 may be further connected to a temperature or flow rate sensor within the cartridge 19 or fluid administration set 18. The information key 29 may be a mechanical device, an electrical device, a magnetic device, an optical device or a combination of these. Referring to FIGS. 1 and 2, the information key 29 is coupled or interfaced to the key interface 37 on the controller. Such key interface 37 may be electrical, magnetic, optical, mechanical or a combination of these. The key interface 37 sends information directly to the a logic circuit, computer or controller within the control unit 1.

Although these are functions supplied by the interaction between the cartridge 19 and the control unit 1, the cartridge 19 will be as simple as possible, and the adaptations and detection mechanisms will chiefly be designed for the control unit 1. This technology must also be gentle enough to deliver all fluids 20, including blood products that are prone to lysis, so the pumping mechanism will operate at low pressure or low shear rates.

The temperature setting will be controlled by an element or key 29 within the fluid cartridge 19 so that the operator can install the cartridge 19 and know that the temperature is predetermined. This is an important safety feature since the device will be used by emergency medical personnel under adverse field conditions. Predetermined settings assure that inadvertent bumps against the control unit 1 will not alter any parameters critical to patient safety. However, there will be cartridges 19 that do not contain a temperature-setting information key 29 so that control units with a specialized data port can receive more complicated instructions from an operator or from other equipment (such as a computer). This information may be provided by color coded cartridges and/or the label 21. The high surface area to volume ratio of the multiple protrusions in the cartridge 19 are designed to assure that appropriate temperature will be achieved at all rates of flow that can be provided by the pumping mechanism 22 and 23.

The majority of the walls of the protrusions 6 and 24 will be heat exchange surfaces that interface between the control unit 1 and the cartridge 19. Full contact of the two temperature elements 6 and 24 is accomplished by a mechanism that engages when the cartridge 19 is inserted into the receptacle 5. Through mechanical and/or magnetic or other means, the temperature elements 6 on the control unit 1 are moved into greater proximity to the heat exchange surface 24 on the cartridge 19 so that upstream pressure on the flexible heat exchange surface 24 of the cartridge 19, as well as pressure from the pumping mechanism 22 in the control unit 1 against the pumping element 23 within the cartridge 19 achieves full contact between the heat exchange surfaces 6 and 24. The temperature of the fluid 20 is monitored by a thermocouple, mechanical key, or other sensor or information key 29 within the fluid cartridge 19 and detected by the key interface 37 within the control unit 1. One mechanism for regulating fluid 20 temperature can be the operation of only as many temperature elements 6 in the heat exchanger as are needed to achieve the desired fluid temperature. This feature also allows redundancy as protection against the failure of individual temperature elements. A more preferred embodiment is a simple on-off logic circuit for the temperature elements that functions by heating when the temperature is too low and not heating when the temperature reaches the desired value. A rate control unit may be desirable to minimize overshoot and keep the system critically damped. The temperature elements 6 can be standard resistive devices such as those fabricated from high-resistance wire such as nickel-chromium or ceramics and which provide Ohmic heating. The heat exchange surface may itself be the heating element to minimize energy loss. Also, various reversible exothermic chemical reactions may be incorporated for energy storage and fast initial warm-up.

The tubing 16 and 25 will not be fundamentally different from other tubing systems in that it encompasses the standard features-standard connectors at both ends 15 and 28, in-line drip chamber 17 for visual verification of flow, roller clamps 26 or other devices to stop flow if necessary by mechanical intervention, and injection port or ports 27 for administration of pharmaceuticals. The tubing 16 and 25 may differ from other fluid administration sets in that the outflow tubing 25 from the cartridge 19 to the patient 30 may be insulated to protect against temperature loss in cases of low fluid flow rates or low ambient temperatures such as can be found in emergency and other field conditions. Insulation may be achieved by external sources, such as an insulated wrap separate from the fluid line, or may be intrinsic to the line such as an embedded tube within a tube with an insulating air layer.

The power source incorporates a rechargeable battery pack 12 for portability and safety during energy outages. The battery 12 is exchangeable to allow continuous use of one control unit 1 with multiple battery packs 12. The control unit 1 will have an extension power cord 36 with adaptor that allows for operation from power sources such as but not limited to a standard wall outlet or a 12-volt system such as those found in emergency vehicles like ambulances.

Operator controls will determine flow rate, heating, and system on/off. Some embodiments of the control unit 1 will have an optional data port that will allow the operator or another device such as a computer to specify the functions of the control unit 1. Such a data port can be an RS-232, USB or the like. For computer-controlled embodiments there could be a cartridge 19, optionally, without specifications for temperature (since the cartridge sets the temperature in all other circumstances) or the computer controls could be set to override the cartridge 19 temperature set key. For verification of which functions are activated, the on/off buttons will be backlit. There are also flow control buttons 7 with indicator arrows to increase or decrease flow from the current rate. The display 10 on the front panel, switches, and indicator lights 8, 9, 11 and others will allow for monitoring of the unit at a glance, and displays of flow and temperature information will be designed for visibility in both high light and low light conditions.

While certain embodiments of the invention have been described, these embodiments have been presented by way of example only, and are not intended to limit the scope of the invention. Indeed, the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms or modifications as would fall within the scope and spirit of the invention. The present invention may be embodied in other specific forms without departing from its spirit or essential characteristics. The described embodiments are to be considered in all respects only as illustrative and not restrictive. All changes which come within the meaning and range of equivalency of the claims are to be embraced within their scope.

We claim:

1. A fluid management system adapted for delivery of fluids for medical purposes at physiologically appropriate temperatures comprising:
a control unit; wherein the control unit further comprises a pump and temperature elements for heating or cooling through heat exchange; and
a fluid administration set wherein the fluid administration set further comprises a cartridge that interfaces with the control unit for the purposes of heating or cooling and pumping of said fluids.

2. The system of claim 1 further comprising an interlocking mechanism that maintains close contact between the cartridge of the fluid administration set and the control unit.

3. The system of claim 1 further comprising a power supply and a rechargeable battery.

4. The system of claim 1 wherein the cartridge comprises a plurality of extensions that interlock with the temperature elements on the control unit for heat exchange.

5. The system of claim 1 wherein said cartridge comprises a pumping mechanism that engages with the pump in the controller and causes the fluid to be pumped at a controlled rate.

6. The system of claim 1 wherein said controller comprises at least one detector for fluid temperature and fluid flow.

7. The control unit of claim 1 wherein the power supply may be plugged into either a standard 110 VAC wall outlet or a 12 Volt DC automotive system.

8. The control unit and fluid administration set of claim 1 further comprising an information key and a key interface that specifies the intended temperature of the fluid to be delivered.

9. The control unit of claim 1 wherein the control unit weighs 20 pounds or less.

10. A method of fluid administration into a mammalian body comprising the steps of:
connecting a fluid filled reservoir to the inflow end of a length of fluid administration tubing;
providing a cartridge at a midpoint of said fluid administration tubing;
inserting said cartridge into a control unit;
inserting the outflow end of the fluid administration tubing into a vascular access site on a mammalian body, or using the outflow end for provision of fluids to a tissue site;
heating or cooling said fluid as it passes through said cartridge and;
pumping said fluid into the patient at a controlled rate;

11. The method of claim 10 wherein said control unit is self-contained.

12. The method of claim 10 further comprising the step of disposing of said cartridge after the fluid administration is completed.

13. The method of claim 10 further comprising the step of selecting the cartridge further comprises a key, wherein the key pre-determines the temperature to which the controller will heat or cool the fluid.

14. The method of claim 10 further comprising the step of pre-filling said reservoir.

15. The method of claim 10 further comprising the step of selecting the desired flow rate of the fluid to be administered.
